(19) United States
(12) Patent Application Publication
(10) Pub. No.: US 2002/0146361 A1
(43) Pub. Date: Oct. 10, 2002

(54) Thermally-activated fragrance dispenser

(76) Inventors: Chris Jeff Gardner, Joplin, MO (US); Brian Patrick Murphy, Joplin, MO (US)

Correspondence Address: GEORGE E STANFORD JR, 34050 LOUISIANA HWY 433, SUITE A, SLIDELL, LA, 70460, US

(21) Appl. No.: 09/829,528

(22) Filed: Apr. 10, 2001

(51) Int. Cl. A61L 9/00

(52) U.S. Cl. 422/306; 422/307

(57) ABSTRACT

A thermally activated device for perfuming the air immediately surrounding an operating electronic computer monitor, including a thermally conductive receptacle adapted to contain therein a thermally activated fragrance material and further adapted to be affixed over and proximate to the waste heat cooling vents of an electronic computer monitor, and a removable receptacle lid cooperating with the receptacle to retain the fragrance material therein. The receptacle includes a heat absorbing base and a closed peripheral sidewall permanently attached thereto. Preferably, the base exterior bottom surface of the base features lateral rectilinear or curvilinear serriform fins to increase thermal absorption of the waste heated air exiting the computer monitor via the cooling vents to the base exterior bottom surface of the thermally conductive receptacle; the receptacle is adapted for placement against the cooling vent by two-sided adhesive mounting pads or hooks and loops fasteners, such as Velcro® strips. The thermally activated fragrance material within the receptacle perfumes the surrounding air through at least one perforation or a grille portion of the receptacle lid. Multiple perforations may be in a distinctive pattern to personalize the dispenser.

### BACKGROUND

1. Field of the Invention

The present invention concerns generally a fragrance dispenser for perfuming, air freshening, or otherwise pleasurably affecting the olfactory senses. In particular, the present invention is directed to a non-electric, non-mechanical, thermally-activated fragrance dispenser for use with a computer monitor, or the like, and attached thereto, wherein waste heat generated by electrical or electronic circuitry attendant to the monitor, upon exiting from the monitor case or housing via monitor cooling vents, is absorbed by the thermally conductive fragrance dispenser receptacle and activates or accelerates perfuming of the fragrance matter contained therein.

2. Description of Related Art

Various devices for perfuming, deodorizing, or otherwise altering the sense of smell of the air surrounding an individual or group have existed for millennia; their utilization is ubiquitous in virtually all examples of modern society. Places of religious worship, automobiles, public restrooms, and bathrooms are just a small sample of the many places where one may find burning or smoldering incense, auto air fresheners hanging from the rear view mirror, air fresheners secreted in bathroom tissue dispensers, attached to doors, or plugged into electrical outlets. Some such devices function in the ambient environment; other devices require heating, e.g. subliming, of the fragrance material and/or mechanical movement of the air proximate thereto, to activate or accelerate perfuming the surrounding air. Heating of the fragrance material often is accomplished by either combustion retardant means or utilization of an electrical heating element configured within the fragrance dispenser.

Some fragrance dispensers urge the flow of air across and through the fragrance material to expedite perfuming the surrounding air with the scent of the fragrance. In some cases, this action is achieved with a mechanical blower; in other cases, the action is achieved by mechanically moving the fragrance material through the air. U.S. Pat. No. 5,148,984 “DEVICE FOR DISPENSING A VAPORIZABLE MATERIAL” is incorporated herein by reference for purposes of indicating the background of the present invention or illustrating the mature state of the art for dispensing the scent of a vaporizable material, in particular, moving the fragrance material through the air by means of attaching the fragrance material holder to a door.

Some fragrance dispensers are intended for personal use, either worn as an adornment to clothing or decorated with appropriate indicia to suggest personalized, individual use of the dispenser. U.S. Pat. No. 4,465,232 “DEVICE FOR CARRYING A SCENTED ELEMENT FOR AFFIXING TO THE BODY, ARTICLES OF CLOTHING OR PACKAGES” and U.S. Pat. No. 4,909,438 “AIR FRESHENER DISPENSER” are incorporated herein by reference for purposes of indicating the background of the present invention or illustrating the mature state of the art for dispensing the scent of a vaporizable material, in particular, illustrating the state of the art in personalized fragrance dispensing adornments and individualized dispensers, respectively.

The present invention is intended for personal use in the vicinity of an operating computer monitor. In the present invention, enlisting the use of waste heat exiting the cooling vents of a computer monitor, typically utilizing a cathode ray tube and attendant electronic circuitry, activates and accelerates perfuming the surrounding air with a fragrance. Such vents typically comprise an array or field of perforations in the top and/or sides of the cabinet or housing of the monitor to expedite the removal of heated air from the vicinity of the monitor's electronic circuitry.

Whether in individual cubicles or in massed, open floor array, many modern workers spend a very large portion of their working day seated before an electronic computer monitor at a computer workstation. For corporate efficacy, these workstations tend to be uniformly unimaginative, devoid of individual personality. The work performed at these workstations may sometimes be creative and exciting, more often the work is repetitious and somewhat boring. In some work situations, fresh air ventilation may be less than optimum, compounding a potential adverse influence on worker performance.

To personalize these workstations and infuse a modicum of pleasure to the workstation task, workers often affix photographs, cartoons, and other personal indicia to the computer monitor. Another addition to workstation personalization and worker pleasure would be the use of a personalized, individual fragrance perfumed into the air immediate to the computer monitor. To improve worker performance and give the worker a sense of individualism and identity, the present invention provides an individual air freshening and perfuming dispenser affixed to the individual workers' computer monitor. No burning, smoldering device or electrically heated device is needed, only the waste heated air exiting the cooling vents of the monitor cabinet. This is very economical, incurring no additional cost of electrical power or incurring the risk of smoke or fire in the work area.

### SUMMARY

An object of the present invention is to provide a thermally activated fragrance dispenser utilizing waste heat venting from an electronic computer monitor to perfume the air adjacent thereat for the individual pleasure of the monitor operating personnel. Another object of the present invention is to provide a thermally activated fragrance dispenser that neither requires connection to an electrical circuit nor consumes additional electrical or other energy resources. Yet another object of the present invention is to provide a thermally activated fragrance dispenser that does not perfume the air until the electronic monitor is energized and expelling waste heat from its cooling vents. A further object of the present invention is to provide a thermally activated fragrance dispenser that neither requires moving parts nor requires moving the fragrance perfuming material through the air. Still another object of the present invention is to provide a thermally activated fragrance dispenser that is simple and economical to install and operate. Another object of the present invention is to provide a thermally activated fragrance dispenser that may utilize a variety of thermally activated fragrance perfuming materials.

### BRIEF DESCRIPTION OF THE DRAWINGS

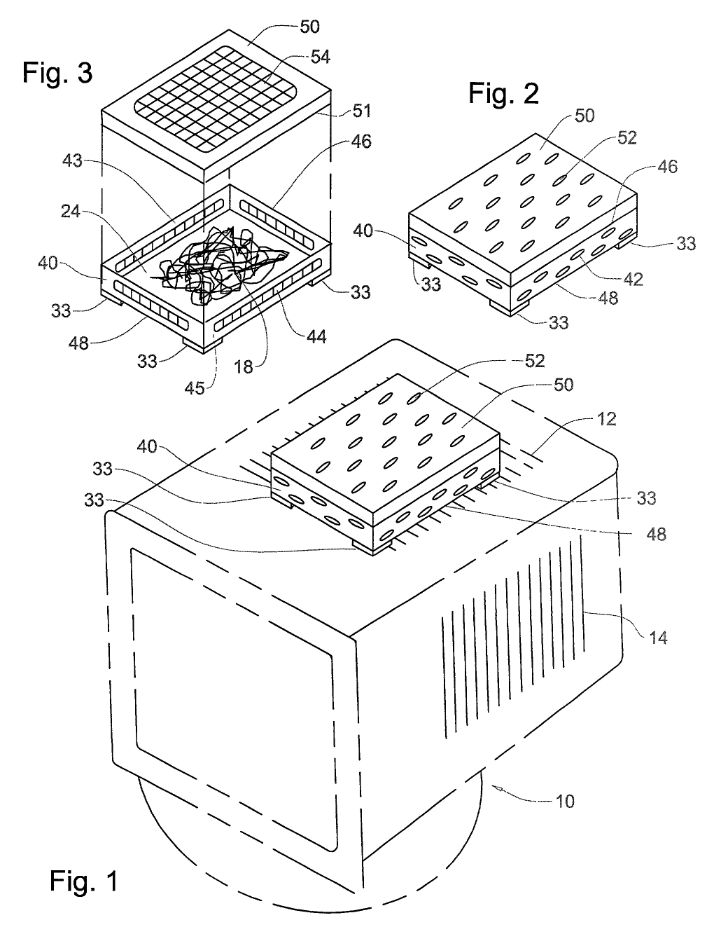

1. FIG. 1 is a perspective view of an embodiment of a thermally activated fragrance dispenser mounted over the top waste heat cooling vents of an electronic monitor, according to the present invention.

2. FIG. 2 is a perspective view of a preferred embodiment of a thermally activated fragrance dispenser featuring perforations in both the lid and sidewall thereof, for use with an electronic monitor, according to the present invention.

3. FIG. 3 is a partially exploded perspective view of an alternative embodiment of a thermally activated fragrance dispenser featuring grilles in both the lid and closed peripheral sidewall thereof, for use with an electronic monitor, according to the present invention. This view is partially exploded to more clearly depict the thermally activated fragrance perfuming material disposed within the closed peripheral sidewall and resting against the interior surface of the heat-collecting base.

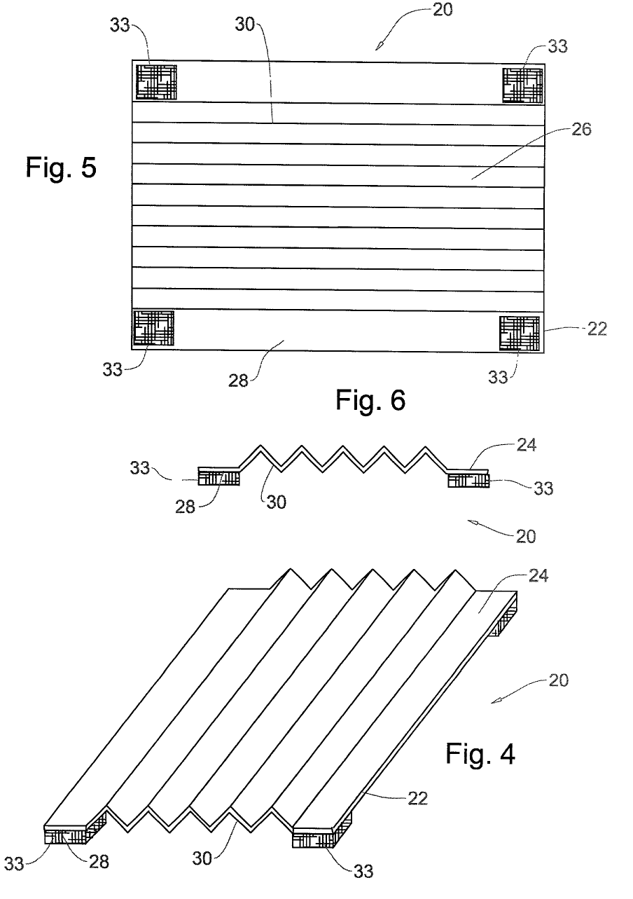

4. FIG. 4 is a perspective view of a preferred embodiment of a thermally conductive base for collecting waste heat exiting the monitor cooling vents and transferring the heat to a thermally activated fragrance perfuming material disposed on the interior surface of the base, according to the present invention. The base illustrated in FIG. 4 is fabricated from a malleable, heat conducting material accordion-folded to produce generally rectilinear serriform topography for the heat absorbing base exterior bottom surface.

5. FIG. 5 is a bottom plan view of FIG. 4 illustrating a preferred embodiment of a thermally conductive base for collecting waste heat exiting the monitor cooling vents and transferring the heat to a thermally activated fragrance perfuming material proximate the interior surface of the base, according to the present invention. Further illustrated is the base exterior bottom surface including a heat-absorbing base exterior bottom surface, exhibiting generally rectilinear serriform topography, and a mounting surface and two-sided adhesive mounting pads attached thereto.

6. FIG. 6 is an end view of FIG. 4 illustrating a preferred embodiment of a thermally conductive base for collecting waste heat exiting the monitor cooling vents and transferring the heat to a thermally activated fragrance perfuming material proximate the interior surface of the base, according to the present invention. Further illustrated is the interior surface and the base exterior bottom surface including a heat-absorbing base exterior bottom surface, exhibiting a generally rectilinear serriform topography, and a mounting surface and mounting pads attached thereto.

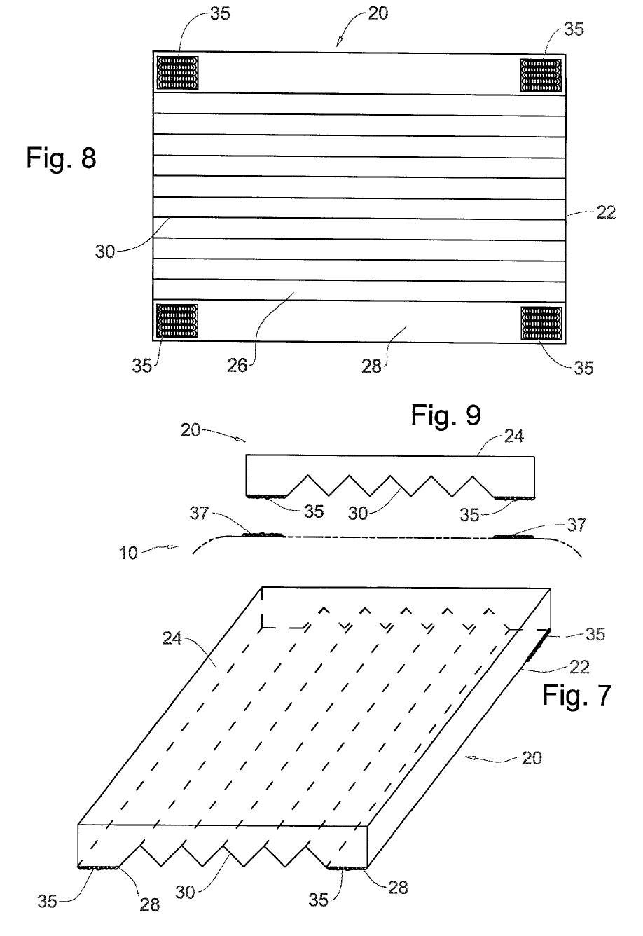

7. FIG. 7 is a perspective view of an alternative embodiment of a thermally conductive base for collecting waste heat exiting the monitor cooling vents and transferring the heat to a thermally activated fragrance perfuming material proximate the interior surface of the base, according to the present invention. The base illustrated in FIG. 7 is fabricated from a heat conducting material cast, molded, extruded, or otherwise modified to produce generally rectilinear serriform topography for the heat-absorbing base exterior bottom surface.

8. FIG. 8 is a bottom plan view of FIG. 7 illustrating an alternative embodiment of a thermally conductive base for collecting waste heat exiting the monitor cooling vents and transferring the heat to a thermally activated fragrance perfuming material proximate the interior surface of the base, according to the present invention. Further illustrated is the base exterior bottom surface including a generally rectilinear serriform heat-absorbing base exterior bottom surface and a mounting surface and a portion of the two part hooks and loops mounting pads attached thereto.

9. FIG. 9 is an end view of FIG. 7 illustrating an alternative embodiment of a thermally conductive base for collecting waste heat exiting the monitor cooling vents and transferring the heat to a thermally activated fragrance perfuming material proximate the interior surface of the base, according to the present invention. Further illustrated is the interior surface and the base exterior bottom surface including a generally rectilinear serriform topography, heat-absorbing base exterior bottom surface and a mounting surface and mounting pads attached thereto.

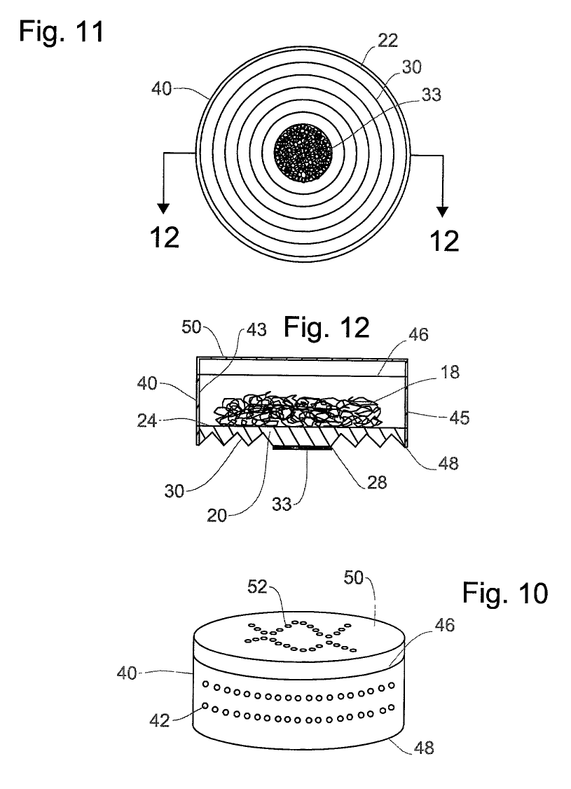

10. FIG. 10 is a perspective view of an alternative embodiment of a thermally activated fragrance dispenser featuring a perforated lid, a perforated sidewall, and a heat-collecting base, all in a non-parallelepiped shape, for use with an electronic monitor, according to the present invention. The perforations may depict a decorative pattern or spell a name or message.

11. FIG. 11 is a bottom plan view of FIG. 10 illustrating an alternative embodiment of a thermally conductive base for collecting waste heat exiting the monitor cooling vents and transferring the heat to a thermally activated fragrance perfuming material proximate the interior surface of the base, according to the present invention. Further illustrated is the base exterior bottom surface including a heat-absorbing base exterior bottom surface, exhibiting generally curvilinear serriform topography, and a mounting surface and two-sided adhesive mounting pads attached thereto.

12. FIG. 12 is a sectional view of FIG. 11 illustrating an alternative embodiment of a thermally conductive base for collecting waste heat exiting the monitor cooling vents and transferring the heat to a thermally activated fragrance perfuming material proximate the interior surface of the base, according to the present invention. Further depicted is the thermally activated fragrance perfuming material, interior surface and the base exterior bottom surface including a generally curvilinear serriform topography heat-absorbing base exterior bottom surface and a mounting surface and two-sided adhesive mounting pad attached thereto.

### DESCRIPTION OF THE PREFERRED EMBODIMENT

The preferred embodiment of a thermally activated fragrance dispenser according to the present invention is generally a parallelepiped receptacle with a lid 50 adapted thereto, the lid 50 and closed peripheral sidewall 40 of the receptacle featuring through openings to permit perfuming the air in the vicinity of the dispenser. In the preferred embodiment, base 20 and closed peripheral sidewall 40 are fixedly conjoined, forming a receptacle having a sidewall interior volume sufficient to receive and contain therein a thermally-activated fragrance perfuming material 18 (not claimed in the present invention). The primary purpose of lid 50 is to securely retain perfuming material 40 within the sidewall interior volume, particularly when the present invention is mounted against a vertical side cooling vent 14 of a monitor 10. Lid 50 need be opened or closed only to remove or replace perfuming material 18; it does not need to be manipulated during normal operational use of the present invention. Consequently, a wide variety of techniques may be employed to attach lid 50 to the receptacle. In the preferred embodiment, the principal component of any combination used to attach and secure lid 50 to the receptacle is a hinge or snap-on crimp, or a combination thereof. In a perspective view, FIG. 1 depicts a thermally activated fragrance dispenser according to the present invention installed atop the top waste-heat cooling vent 12 of electronic computer monitor 10. An alternative installation could posit the present invention against the side waste-heat cooling vent 14. In either installation, waste-heat exiting the cooling vents when monitor 10 is energized is absorbed within the present invention.

The collected thermal energy is transferred thereupon to a thermally activated fragrance perfuming material 18 (not claimed in the present invention) disposed within the present invention. Perfuming of the air adjacent monitor 10 occurs as the warmed fragrant air within the present invention exits the dispenser via a plurality of sidewall perforations 42 of closed peripheral sidewall 40 and a plurality of lid perforations 52 of lid 50, as illustrated in perspective views FIG. 1 and FIG. 2. Perforations 42 and 52 can be arranged to form a decorative pattern or present a text, such as a name, event, or phrase.

Typically, the present invention is installed over and proximate to either the top or side cooling vents by means of at least one two-sided adhesive mounting pad 33. Alternatively, installing the present invention on the cooling vents 12 and 14 could be achieved with at least one two-part conjoined hooks and loops mounting pad, such as Velcro®, a first hooks and loops mounting pad 35 affixed to the present invention and a second hooks and loops mounting pad 37 affixed to the monitor 10.

An alternative means for perfuming the air adjacent monitor 10 utilizes a lid grille 54 of lid 50 and a sidewall grille 44 of closed peripheral sidewall 40 instead of lid perforations 52 and sidewall perforations 42, respectively. This alternative variation is shown in FIG. 3, which further illustrates closed peripheral sidewall 40 having a sidewall interior surface 43 and a sidewall exterior surface 45. FIG. 3 is a partially exploded view to clarify depiction of thermally activated fragrance material 18 (not claimed in the present invention) resting on base interior surface 24 of heat absorbing base 20. When the present invention is completely assembled and is in a state of operational readiness, lid peripheral edge 51, of lid 50, matches the geometry and rests proximate to upper peripheral edge 46 of closed peripheral sidewall 40.

FIGS. 4, 5, 6 illustrate, in more detail, the preferred embodiment of the heat absorbing base 20 of the present invention. The preferred material for base 20 is metal. In the preferred embodiment, heat absorbing base 20 is fabricated from a malleable, thermally conductive material, accordion-folded to produce a heat-absorbing base exterior bottom surface 30, exhibiting a generally rectilinear serriform topography, upon base exterior bottom surface 26. At least one portion of base exterior bottom surface 26 is configured for use as a mounting surface 28, to receive at least one two-sided adhesive mounting pad 33 or alternately, at least one portion of a dual hooks and loops mounting pad, comprising a first hooks and loops mounting pad 35 and a second hooks and loops mounting pad 37.

FIG. 5, a bottom plan view of base 20 depicts the projected area, or “footprint”, bounded by base periphery 22. During intended use of the thermally activated dispenser, the “footprint” is the base area of the present invention covering a portion of one of the waste-heat cooling vents of monitor 10. It is readily appreciated, that for a given “footprint”, the heat-absorbing base exterior bottom surface 30, exhibiting a generally rectilinear serriform topography in the preferred embodiment of the present invention, of base 20 substantially increases the effective heat absorbing surface area, and consequently increases the efficacy of the transfer of thermal energy from the cooling vents to thermally activated fragrance perfuming material 18 (not claimed in the present invention).

To form an appropriate receptacle for perfuming material 18, lower peripheral edge 48 of closed peripheral sidewall 40 is adaptively and permanently conjoined by conventional means, e.g. solder, glue, crimping, to base periphery 22 of base 20. Many combinations could be utilized; however, in the preferred embodiment the principal component of any such combination is epoxy resin. To retain the perfuming material within the receptacle, lid 50 is adapted to fit the general shape of upper peripheral edge 46 of sidewall 40.

A variant of the preferred embodiment of the present invention is use of a thermally conductive material for heat absorbing base 20, where the material is not malleable. In this variation, shown in FIGS. 7, 8, 9, the material for base 20 is forged, pressed, cast, extruded, molded, or machined to produce, from base exterior bottom surface 26, a heat-absorbing base exterior bottom surface 30, exhibiting a generally rectilinear serriform topography in an alternative embodiment of the present invention, and at least one mounting surface 28. The interior surface 24 may be flat. FIGS. 7, 8, 9 also display a variation in the method for installing the fragrance dispenser against the cooling vents of monitor 10. In this instance, a two-part hooks and loops mounting pad, such as Velcro®, comprises a first hooks and loops mounting pad 35, adhesively affixed to mounting surface 28, and a second hooks and loops mounting pad 37, adhesively affixed to top cooling vent 12 or side cooling vent 14 of monitor 10. The fragrance dispenser according to the present invention is installed upon the cooling vents of monitor 10 by conjoining pad 35 and pad 37.

Although the general shape of the preferred embodiment of the present invention is parallelepiped, other shapes are not precluded. FIGS. 10, 11, 12 illustrate an alternate embodiment of the fragrance dispenser according to the present invention. FIG. 10 is a perspective view of a non-parallelepiped version of the present invention. The plurality of sidewall perforations 42 and lid perforations 52 in sidewall 40 and lid 50, respectively, enable perfuming of the air proximate monitor 10 from the thermally activated fragrance perfuming material heated within the sidewall 40 and lid 50 of the present invention. The perforations may define a decorative pattern or otherwise provide a text message or name. The general shape of the alternate embodiment need not be symmetrical.

FIG. 11 is a bottom plan view of an alternate embodiment of the present invention. Shown are the base periphery 22, the heat-absorbing base exterior bottom surface 30, featuring a generally curvilinear serriform topography in the alternate embodiment, and the two-sided adhesive mounting pad 33. FIG. 12 is a sectional view of FIG. 11, illustrating the thermally activated fragrance perfuming material 18 resting on interior surface 24 of base 20. Lower peripheral edge 48 of closed peripheral sidewall 40 is adaptively and fixedly conjoined with base periphery 22, forming a receptacle to contain therein, perfuming material 18. Attributes of closed peripheral sidewall 40 include sidewall interior surface 43, sidewall exterior surface 45 and a sidewall interior volume. Lid 50 is adapted to fit the geometry of upper peripheral edge 46 to further contain perfuming material 18 within the present invention. Also shown in FIG. 11 is the two-sided adhesive mounting pad 33, affixed to mounting surface 28. An alternative fastening method for fastening the present invention to computer monitor 10 could be the employment of hooks and loops fasteners, such as Velcro®.

Although only a few exemplary embodiments of the invention have been described in detail above, those skilled in the art will readily appreciate that many modifications are possible in the exemplary embodiments without materially departing from the novel teachings and advantages of this invention. Accordingly, all such modifications are intended to be included within the scope of this invention as defined in the following claims. In the claims, means-plus-functions clauses are intended to cover the structures described herein as performing the recited functions and not only structural equivalents but also equivalent structures.

What is claimed is:

1. A thermally-activated fragrance dispenser for perfuming the air in the vicinity of an energized, waste-heat vented, electronic computer monitor, said dispenser comprising:
a heat-absorbing, thermodynamic base, composed of a thermally conductive material, said base having a base periphery defined by a top plan-view thereof, a base interior surface, and a base exterior bottom surface, said base exterior bottom surface including at least one mounting surface and at least one heat-absorbing base exterior bottom surface;
a closed peripheral sidewall, said sidewall having a sidewall interior volume, a closed sidewall interior surface and a closed sidewall exterior surface parallel thereto, said sidewall having at least one sidewall perforation therein, piercing said closed sidewall exterior surface and said closed sidewall interior surface, said sidewall having a closed upper peripheral edge defined by a top plan-view thereof and a closed lower peripheral edge parallel and diametric thereto, the geometry of said closed upper peripheral edge and the geometry of said closed lower peripheral edge generally conforming to the geometry of said base periphery, said closed lower peripheral edge of said sidewall and said base periphery of said base each adapted to be fixedly conjoined, for receiving and holding a thermally-activated fragrance material;
a fragrance dispenser lid, having at least one lid perforation therein, said lid having a lid peripheral edge, the geometry of said lid edge defined by a top plan-view thereof and generally conforming to the geometry of said closed upper peripheral edge of said sidewall, said lid peripheral edge and said upper edge of said sidewall each adapted to be movably conjoined, for receiving and securely containing therein the thermally-activated fragrance perfuming material;
means for increasing the thermal absorption and transfer properties of said base, for promoting thermal activation of the fragrance perfuming material disposed thereon;
means for fixedly conjoining said lower peripheral edge of said sidewall with said base periphery of said base;
means for movably conjoining said lid peripheral edge with said closed upper peripheral edge of said sidewall, for convenient access to the sidewall interior volume of said closed peripheral sidewall for disposing the fragrance perfuming material therein;
means for installing said dispenser over the waste-heat vent of the monitor; and
means for promoting perfuming the air in the vicinity of the energized, waste-heat vented, electronic computer monitor;
said dispenser adaptively installed over the waste-heat vent of the monitor, the monitor having at least one waste-heat vent to expel waste heat generated by the energized electronic portion of the monitor, said dispenser having the thermally-activated fragrance perfuming material disposed therein, the perfuming material in thermal contact with said interior surface of said base, waste heat expelled from the waste-heat vent of the energized monitor absorbed by said heat absorbing base exterior bottom surface, thermally transferred to said interior surface, the waste heat thereupon transferred to the thermally-activated fragrance perfuming material, the thermally-activated perfuming fragrance exiting said dispenser through said at least one sidewall perforation and said at least one lid perforation, for perfuming the air in the vicinity of the energized, waste-heat vented, electronic computer monitor.

2. A thermally-activated fragrance dispenser as recited in claim 1, wherein said means for fixedly conjoining said lower peripheral edge of said sidewall with said base periphery of said base is selected from a group consisting of solder, epoxy resin, glue, welding, rivets, screws, and crimp tabs.

3. A thermally-activated fragrance dispenser as recited in claim 2, wherein said means for movably conjoining said lid peripheral edge with said upper peripheral edge of said sidewall is selected from a group consisting of hinge, snap-on crimp, threaded screw, and snap fastener.

4. A thermally-activated fragrance dispenser as recited in claim 3, wherein said means for promoting perfuming the air in the vicinity of the energized electronic monitor comprises a plurality of said sidewall perforations in said sidewall and a plurality of said lid perforations in said lid, said plurality of sidewall perforations and said plurality of lid perforations each selected from a group consisting of a grille, perforations expressing a decorative ornamentation, perforations expressing a message, and perforations expressing a name.

5. A thermally-activated fragrance dispenser as recited in claim 4, wherein said means for installing said dispenser over the waste-heat vent of the energized electronic monitor comprises at least one mounting pad affixed to said at least one mounting surface of said base, adapted for conjoining said thermally-activated fragrance dispenser with the monitor waste-heat vent, said at least one mounting pad selected from a group consisting of a two-part mounting strip, each part an adhesively-backed, hooks and loops strip, such as Velcro®; a two-sided adhesive strip; epoxy resin; rivets; screws; and glue.

6. A thermally-activated fragrance dispenser as recited in claim 5, wherein said means for increasing the thermal absorption and transfer properties of said base comprises said base fabricated from malleable heat-conducting material, crimped in accordion-fold, producing said base exterior bottom surface having said at least one mounting surface and said at least one heat-absorbing base exterior bottom surface having a generally serriform topography for increasing the heat absorbing surface area of said heat-absorbing base exterior bottom surface of said base for promoting thermal activation of the thermally-activated fragrance perfuming material.

7. A thermally-activated fragrance disperser as recited in claim 5, wherein said means for increasing the thermal absorption and transfer properties of said base comprises said base fabricated from a block of heat-conducting material, said block, upon fabrication, having for said base exterior bottom surface said at least one mounting surface and said at least one heat-absorbing base exterior bottom surface having a generally serriform topography for increasing the heat absorbing surface area of said heat-absorbing base exterior bottom surface of said base, material for said base selected from a group consisting of metal, thermally conducting ceramic, thermally conducting glass, and thermally conducting plastic.

8. A thermally-activated fragrance dispenser for perfuming the air adjacent an energized, waste-heat vented, electronic computer monitor, said dispenser comprising:
a heat-absorbing base, composed of a thermally conductive material, said base adapted to be fixedly mounted over the waste-heat vent of the vented electronic computer monitor, said base having a base periphery defined by a top plan-view thereof, a base interior surface, and an base exterior bottom surface, said base exterior bottom surface including at least one mounting surface and at least one heat-absorbing base exterior bottom surface, said heat absorbing surface including means for maximizing heat absorption;
a closed peripheral sidewall, said sidewall having a sidewall interior volume, a closed sidewall interior surface and a closed sidewall exterior surface parallel thereto, said sidewall having at least one sidewall opening therein, piercing both said closed sidewall exterior surface and said closed sidewall interior surface, said sidewall having a closed upper peripheral edge defined by a top plan-view thereof and a closed lower peripheral edge diametrically opposite said closed upper peripheral edge, said closed upper peripheral edge defining an upper edge plane, said closed lower peripheral edge defining a lower edge plane, the upper edge plane, the lower edge plane, and said closed sidewall interior surface defining the sidewall interior volume, the geometry of the upper peripheral edge plane and the geometry of the lower peripheral edge plane generally conforming to the geometry of said base periphery, said closed lower peripheral edge of said sidewall and said base periphery of said base each adapted to be fixedly conjoined, for receiving and containing a thermally-activated fragrance perfuming material within the sidewall interior volume; and
a fragrance dispenser lid, having at least one lid opening therein, said lid having a lid peripheral edge, said lid edge geometry defined by a top plan-view thereof, said lid peripheral edge adapted to be movably conjoined with said upper edge of said sidewall, for receiving and securely holding the thermally-activated fragrance perfuming material within the sidewall interior volume;
said dispenser installed over the computer monitor waste-heat vent, said heat absorbing base exterior bottom surface of said base exterior bottom surface proximate thereto, the monitor having at least one waste-heat vent to expel waste heat generated by the energized electronic portion of the monitor, said dispenser having the thermally-activated fragrance perfuming material disposed therein, the perfuming material in thermal contact with said interior surface of said base, waste heat expelled from the waste-heat vent of the energized monitor absorbed by said heat absorbing surface, thermally transferred to said interior surface, the waste heat thereupon transferred to the thermally-activated fragrance perfuming material, the thermally-activated fragrance exiting said dispenser through said at least one sidewall opening and said at least one lid opening, for perfuming the air adjacent the energized, waste-heat vented, electronic computer monitor.

9. A thermally-activated fragrance dispenser as recited in claim 8, wherein said at least one sidewall opening in said sidewall and said at least one lid opening in said lid are adapted to promote perfuming the air adjacent the energized electronic monitor, said at least one sidewall opening in said sidewall and said at least one lid opening in said lid are each selected from a group consisting of a grille, perforations expressing a decorative ornamentation, perforations expressing a message, and perforations expressing a name.

10. A thermally-activated fragrance dispenser as recited in claim 9, wherein the principal component attendant to fixedly conjoining said lower peripheral edge of said sidewall with said base periphery of said base is selected from a group consisting of solder, epoxy resin, glue, welding material, brazing material, rivets, screws, and crimp tabs.

11. A thermally-activated fragrance dispenser as recited in claim 10, wherein the principal component attendant to movably conjoining said lid peripheral edge with said upper peripheral edge of said sidewall is selected from a group consisting of a hinge, a snap-on crimp, and a snap fastener.

12. A thermally-activated fragrance dispenser as recited in claim 11, wherein the principal component attendant to installing said dispenser over the waste-heat vent of the energized electronic monitor comprises at least one mounting pad affixed to said at least one mounting surface of said base, adapted for conjoining said thermally-activated fragrance dispenser with the monitor waste-heat vent, said at least one mounting pad selected from a group consisting of a two part mounting strip, each part an adhesive-backed hooks and loops strip, such as Velcro®; a single two-sided adhesive strip; epoxy resin; and glue.

13. A thermally-activated fragrance disperser as recited in claim 12, wherein said base is fabricated from malleable heat-conducting material, crimped in accordion-fold, producing said base exterior bottom surface having said at least one mounting surface and said heat absorbing base exterior bottom surface having a generally serriform property for increasing the heat absorbing surface area of said base exterior bottom surface of said base for promoting thermal activation of the thermally-activated fragrance perfuming material disposed within said dispenser.

14. A thermally-activated fragrance dispenser as recited in claim 12, wherein said base is fabricated from a block of heat-conducting material, said fabricated block, having for said base exterior bottom surface said at least one mounting surface and said heat absorbing base exterior bottom surface, upon fabrication, having a generally serriform property for increasing the heat absorbing surface area thereof, material for said base selected from a group consisting of metal, thermally conducting glass, thermally conducting ceramic, and thermally conducting plastic.

15. A thermally-activated fragrance dispenser for use with an electronic computer monitor, said dispenser comprising:
a thermally conductive base receptacle adapted to support thereon a thermally activated fragrance element, said base adapted to provide an base exterior bottom surface having features thereto for expeditiously absorbing heat and transferring heat to the thermally activated fragrance element, said base further adapted to be supported overlaying the waste-heat vent of the monitor; and
a receptacle lid supported on said base receptacle and cooperating with said base receptacle to confine therein the thermally activated fragrance element, said base receptacle and said receptacle lid each having at least one perforation therethrough for perfuming the air immediately surrounding the electronic computer monitor during energizing thereof.

16. A thermally-activated fragrance dispenser as recited in claim 15, wherein a plurality of said at least one perforation form a pattern, the pattern selected from a group consisting of a grille, perforations expressing a decorative ornamentation, perforations expressing a message, and perforations expressing a name.

17. A thermally-activated fragrance dispenser as recited in claim 16, wherein said base exterior bottom surface is non-planar for maximizing the heat absorbing characteristics thereof.

18. A thermally-activated fragrance dispenser as recited in claim 17, wherein said base exterior bottom surface is fabricated from malleable heat-conducting material, crimped in accordion-fold, producing said base exterior bottom surface having a generally serriform property for increasing the heat absorbing surface area of said base exterior bottom surface of said base for promoting thermal activation of the thermally-activated fragrance perfuming element.

19. A thermally-activated fragrance dispenser as recited in claim 17, wherein said base exterior bottom surface is fabricated from a block of heat-conducting material, said block, upon fabrication, having a generally serriform property for increasing the heat absorbing surface area of said base exterior bottom surface, heat-conducting material thereof selected from a group consisting of metal, thermally conductive ceramic, thermally conductive glass, and thermally conductive plastic.
